(19) United States
(12) Patent Application Publication
(10) Pub. No.: US 2024/0189491 A1
(43) Pub. Date: Jun. 13, 2024

(54) EMBOLIZATION WITH TRANSIENT MATERIALS

(71) Applicant: Incept, LLC, Lexington, MA (US)

(72) Inventors: Amarpreet S. Sawhney, Lexington, MA (US); Timothy S. Jarrett, Boston, MA (US); Hans Claesson, Lexington, MA (US)

(21) Appl. No.: 18/584,895

(22) Filed: Feb. 22, 2024

Related U.S. Application Data
(63) Continuation of application No. 16/870,473, filed on May 8, 2020.
(60) Provisional application No. 62/846,464, filed on May 10, 2019.

(51) Int. Cl. A61L 33/00; A61L 33/08; A61M 25/09

(52) U.S. Cl. CPC A61L 33/0047; A61L 33/0064; A61L 33/08; A61M 25/09; A61M 2025/091; A61M 2202/0413; A61M 2202/0466; A61M 2210/083; A61M 2210/086; A61M 2210/1003; A61M 2210/12

(57) ABSTRACT

Use of embolic material that is biodegradable provides for embolizing a hypervascular vessel formed in response to chronic inflammation in a musculoskeletal vasculature or a vessel related to production of ghrelin. The embolic material is biodegradable within a predetermined period of time. Medical systems are configured for delivery of embolic material for embolizing the hypervascular vessel or the vessel related to production of ghrelin.

### CROSS REFERENCE TO RELATED APPLICATIONS

This application is a continuation of copending U.S. patent application Ser. No. 16/870,473 filed May 8, 2020 to Sawhney et al., entitled “Embolization with Transient Materials,” which claims priority to U.S. provisional patent application 62/846,464 to Sawhney et al., entitled “Embolization with Transient Materials,” both of which are incorporated herein by reference.

### TECHNICAL FIELD

The technical field is materials and methods of embolization, particularly for treating hypervascularity in response to chronic inflammation.

### BACKGROUND

Osteoarthritis (OA) is a common degenerative joint disease. It is characterized by pain and is generally accepted to be an inflammatory disease of synovial joints. Neovascularization can result from the chronic inflammation and contributes to further inflammation that may lead to pain and further degradation of the joint. Okuno et al., J Vasc Interv Radiol (2017) 28:995-1002.

### SUMMARY

Disclosed herein are materials and methods for embolization of a neovascularization or other blood vessel or a lumen with transient materials. These materials and methods include using an embolic that is fully biodegraded in a period of time in a range from 15 minutes to 48 hours. The materials biodegrade so that flow is restored to normal vasculature and no further intervention is needed. The transient nature of the embolic is in contrast to permanent embolization, nonpermanent embolization for an uncontrolled period of time, or to materials that are biodegradable over longer times.

In a first aspect, the invention pertains to use of an embolic material in a method for embolizing a hypervascular vessel formed in response to chronic inflammation in a musculoskeletal vasculature, in which the use comprises advancing a catheter through a vasculature to a parent artery and releasing the embolic material from a distal end of the catheter into a hypervascular vessel, with the embolic material blocking blood flow in the hypervascular vessel. The embolic material can be biodegradable within a predetermined period of time.

In a further aspect the invention pertains to use of embolic material in a method for embolizing a vessel related to production of ghrelin, in which the method comprises advancing a catheter through a vasculature to a parent artery; and releasing the embolic material from a distal end of the catheter into a vessel related to production of ghrelin, with the embolic material blocking blood flow in the vessel. The embolic material is biodegradable within a predetermined period of time.

In another aspect the invention pertains to a medical system configured for performing the use involving the embolization of a hypervascular vessel formed in response to chronic inflammation in a musculoskeletal vasculature or the vessel related to production of ghrelin. The medical system comprises a catheter and a delivery component comprising a reservoir of embolic material configured for delivery through the catheter.

In other aspects, the invention pertains to a medical system for treatment of hypervascular vessel formed in response to chronic inflammation in a musculoskeletal vasculature or of a vessel related to production of ghrelin, in which the medical system comprises a catheter and a delivery component. The catheter can be suitable for delivery through a patient's vasculature to reach the hypervascular vessel or the vessel related to production of ghrelin. The delivery component can comprise a reservoir of embolic material and a delivery device configured to deliver the embolic material through the catheter.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts a catheter system suitable for the delivery of embolic material to practice the methods of the invention.

FIG. 2A depicts a guide wire positioned in a branch artery.

FIG. 2B depicts a catheter shaft which has been introduced over the guide wire as positioned at a branch artery as shown in FIG. 2A.

FIG. 2C depicts the release of embolization beads from the catheter shaft positioned as shown in FIG. 2B.

FIG. 2D depicts the release of embolization beads from the catheter shaft and the bridging of beads across the branch artery.

FIG. 2E depicts an embolus in a branch artery.

### DETAILED DESCRIPTION

Materials and methods are disclosed herein for the embolization of vasculature with a transient embolic effect and includes embolization for treating hypervascularity in response to chronic inflammation.

A hypervascularized tissue is characterized by a network of blood vessels that starts as a branch from an artery that is normal in appearance. The branch gives rise to further branches and/or fine blood vessels. The fine blood vessels are visualized as a “blush” on an angiogram using radiopaque compounds for visualization in a manner customary in these arts. Without being bound to a particular theory, it is believed that the elimination of the fine vessels is typically adequate for treatment of pain associated with hypervascularization and it is not necessary to place embolics into the largest of the branches. Therefore, treatments can be directed to avoiding embolization of the relatively large branches while embolizing the fine branches. Undesirable side effects that result from targeting the relatively larger branches can then be avoided. As a part of this theory, fast-degrading materials are used to embolize fine vasculature so that there is no, or little, recanalization, i.e., the effects of embolization are permanent. The same fast-degrading materials may temporarily embolize larger vessels without compromising the efficacy of the treatment and also without causing harmful side effects that result from treatments that target the relatively larger vessels. Further, materials may be used that are biodegradable to leave only biocompatible residues, which is a term used herein that means residues of an embolic material that are soluble components that can be locally cleared by dissolution into blood and eventually systemically cleared over time by excretory mechanisms.

Adverse events possible with embolics that are sized to target the relatively larger vessels include accidental embolization of off-site blood vessels with unwanted effects that range from minimal to catastrophic. Others that have used methods that embolize the relatively larger vessels in the context of treating hypervascularity in response to chronic inflammation have observed harmful side effects such as skin necrosis/color change, peripheral paresthesia/numbness, and one or more of muscle weakness, dullness, and pain. These unwanted and harmful side effects may be reduced or eliminated with certain embodiments of the invention described herein.

An embodiment of the invention is an embolization technique that involves biodegrading embolics that biodegrade within certain periods of time and/or embolics that fall within certain size ranges. Biodegradation can be characterized by in vitro methods or in vivo methods. Particles are useful embolic materials. Microspheres have some advantages in mechanical and fluid flow properties.

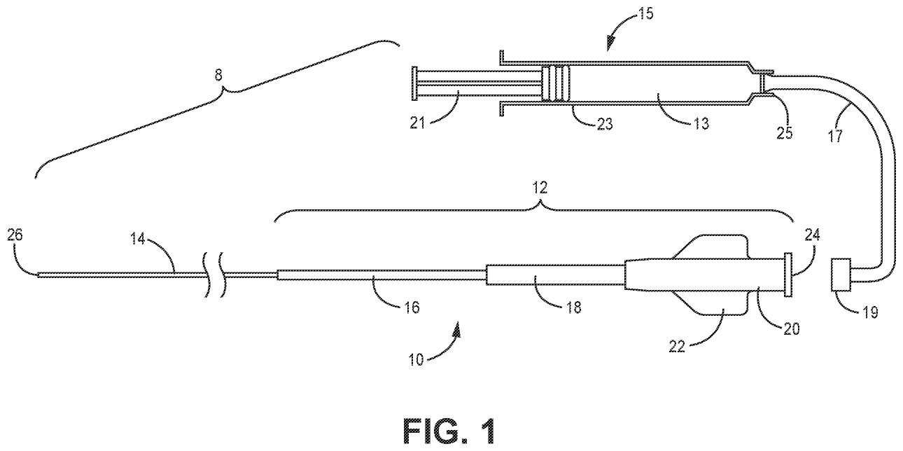

A technique to deliver embolics is with a catheter system 8. FIG. 1 depicts catheter 10 having hub assembly 12 and shaft 14. Hub assembly 12 has intermediate portion 16, strain relief member 18, and hub 20 with hub wings 22 and proximal hub connecter 24. Shaft 14 has distal outlet tip 26. Strain relief member 18 provides a transition from flexible shaft 14 to hub 20. Intermediate portion 16 is optional and may be provided as a further strain relief member over shaft 14 and/or as a portion of shaft 14 that has a large inner diameter (ID) and/or outer diameter (OD). Referring to FIG. 1, catheter system 8 also comprises embolics delivery components having an embolics reservoir 13 and a delivery device 15, such as a syringe or a pump, flow tubing or the like 17 and a generally a connector 19, such as a luer fitting for attachment to proximal hub connector 24. As shown in FIG. 1, delivery device 15 is a syringe with plunger 21, barrel 23 and connector 25 for attachment of flow tubing 17. Artisans are familiar with these components and their operation, as well as their introduction and use in cooperation with guide wires, hemostatic introducers and other components for catheter procedures.

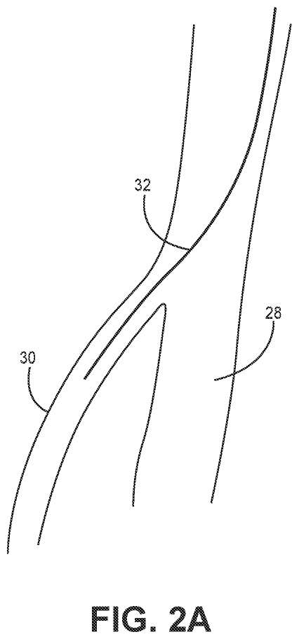

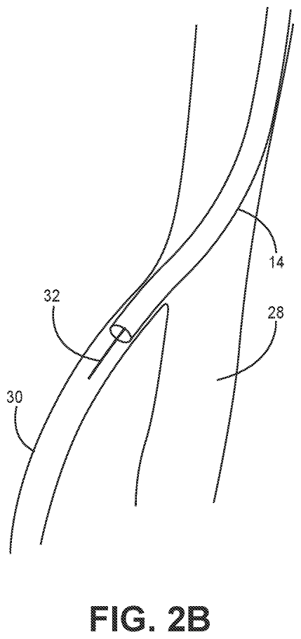

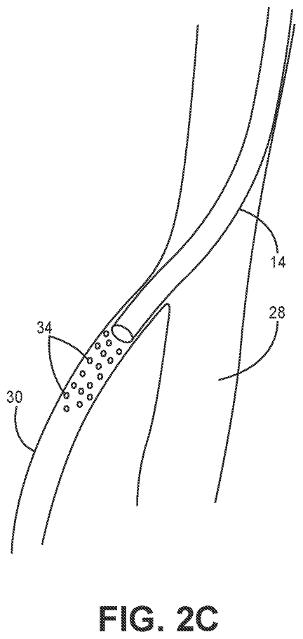

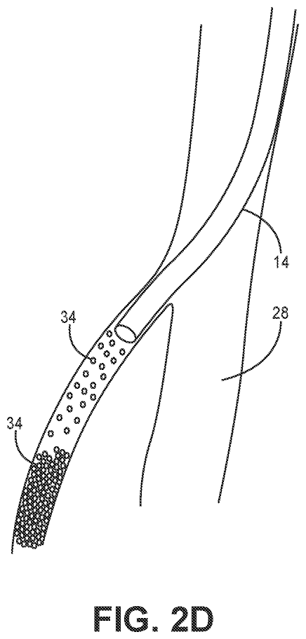

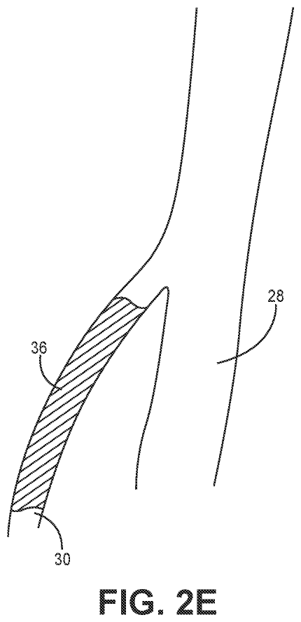

Embolization may be performed by placing a guide wire at a desired position, as at FIG. 2A that depicts artery 28 with branch artery 30, with guide wire 32 positioned in branch 30. Referring to FIGS. 2B-2E, catheter shaft 14 is introduced over guide wire 32 and positioned at a target vasculature such as branch artery 30. An embolizing material, for example embolization beads 34, is injected through a catheter shaft 14. Beads 34 bridge across artery branch 30 to form embolus 36 that blocks blood flow and catheter shaft 14 is withdrawn.

Others have reported that embolization of a geniculate artery and/or a neovascular vessel pending from a geniculate artery is useful to relieve pain in knee joints of mild and/or early stage osteoarthritic joints. It is theorized that hypervascularization of the knee joint provides inflammatory cells and agents increased access to the joint to cause. Okuno et al. (2017) reported results of experiments using embolization of neovascular vessels of geniculate arteries with 75-μm polymethylmethacrylate microspheres with a coat of polyzene-F (EMBOZENE) or with Imipenem/cilastatin sodium (IPM/CS; PRIMAXIN; Merck, Whitehouse Station, New Jersey) in iodinated contrast medium (HEXABRIX; Terumo, Tokyo, Japan). IPM/CS is reported to be a crystalline compound that is slightly soluble in water and forms particles with an embolic effect when it is suspended in contrast medium. The terms “permanent” or “nonbiodgradable” in these contexts means that the embolic materials persist as a recognizable mass in the location where they are placed as embolics for at least 5 years when used for embolization in a patient. In fact, such materials will normally last longer than the patient's lifetime.

Permanent embolization has some disadvantages, such as necrosis and being permanent and irreversible. IPM/CS is not a permanent embolic material. However, the size and shape of particles that are formed by IPM/CS is unclear and not well controlled. Further, the IPM/CS particles are believed to be rigid, non-swelling, and potentially may provide inconsistent blockage of blood flow since the particles are not necessarily optimized to pack together in a way that prevents channeling of fluid through the embolus. Further, IPM/CS is needlessly bioactive since it is primarily an antibiotic and no antibiotic effect is called for in an embolization of a geniculate artery. Also, delivery of a small dose of an antibiotic is disfavored since it promotes a development of microbial resistance.

Further, unwanted embolization at off-target locations can be a challenge. Permanent embolic materials can have permanent effects. Further, in some circumstances, there is reflux, and an embolic component can flow back into a main artery proximal to a distal tip of a delivery catheter, thus carrying an embolic component to unknown and off-target vasculature. As a result, a number of vessels feeding the skin can be embolized leading to numbness and discoloration, which are adverse effects.

An embodiment of the invention is a method of transiently blocking a flow of blood in a musculoskeletal vasculature demonstrating hypervascularity in response to chronic inflammation comprising advancing a catheter through a vasculature to a parent artery; releasing an embolic material from a distal end of the catheter into a hypervascular vessels, with the embolic material blocking blood flow to the hypervascular inflammatory vasculature. The embolic material is biodegradable within the vasculature, or as measured by an in vitro test that relates to physiological conditions, within a time ranging from 15 minutes to 48 hours.

Sheth et al. J. Funct. Biomater. 2017, 8, 12, provides a survey of endovascular embolization by transcatheter delivery of particles. The authors observed that each category of embolic agent is characterized by its respective strengths and weaknesses and enjoys several niche clinical scenarios for which it is ideally suited. They report that PVA particles adhere to blood vessel walls, slowing blood flow and triggering thrombus, and inducing an inflammatory response characterized by angionecrosis of the vessel wall. PVA is not biodegradable but recanalization of vessels embolized with PVA can occur as a result of angiogenesis within the thrombus. Gelation sponge is another material that has been used as a transcatheter embolic agent; it is biodegradable but induces thrombosis and causes a necrotizing arteritis reaction. The authors report that commercially available microspheres generally were composed of PVA, trisacryl-gelatin, polymethylmethacrylate microspheres with a coat of polyzene-F, and QUADRASPHERE super-absorbent copolymer. Significantly, they report that even within an identical size range, microspheres of different formulations have different adhesivity, aggregation behavior, and will embolize vessels at different levels of the vascular tree.

In contrast to such microspheres, a useful embolization material is starch microspheres, for example as in U.S. Pat. No. 4,124,705 or amilomer, which is a generic name (INN name) for certain degradable starch spheres. Amilomer is a synthetic microsphere formulation with arterial occlusive properties. Amilomer, a product produced by partially hydrolyzing starch and epichlorohydrin, contains degradable starch microspheres with a diameter size of 45 micrometers that are readily degraded by amylase. When used in transcatheter arterial chemoembolization (0) procedures, infusion leads to lodging of the microspheres in the precapillary vessels and, consequently, to the occlusion of the hepatic artery.

An embodiment of starch microspheres is EMBOCEPT S DSM 35/50 (Pharmacept) which is a short-term embolic that is composed of degradable starch microspheres with an average diameter of 50 micrometers. The microspheres are enzymatically degraded by serum alpha-amylases in the blood, yielding a half-life of approximately 35-50 minutes, both in vivo and in vitro. The reticulocyte system clears the starch fragments. With degradable starch microspheres, partial resumption of blood flow is observed after approximately 10 to 15 minutes Schicho et al., Oncotarget (2017) 8:72613-72620.

Starches that may be used include a polysaccharide built up of glucose units incorporated (as such or in the form of a physiologically acceptable derivative) in cross linked form in the particles, and that are capable of being degraded by d-amylase into water-soluble fragments, i.e. the polysaccharide shall contain a (1-4) glucosidic linkages which are hydrolyzable by α-amylases. Examples of such polysaccharides include primarily starch and glycogen or dextrins thereof. The starch may be amylose or amylopectin or mixtures thereof. Other glucose-containing polysaccharides which can be hydrolyzed by a amylase can also be used, in connection with which said polysaccharides may be synthetic or may be obtained from biological material, for example from microorganisms. The starch may have a number of repeated glucose subunits (n) in a range of 300 to 1,000,000; Artisans will immediately appreciate that all ranges and values between the explicitly stated bounds are contemplated.

The amylose or other starch in an embolic particle or microsphere is crosslinked, preferably with covalent bonds. Crosslinking of starch may be performed with epichlorohydrin or other crosslinking agents. Methods for crosslinking starch may include use of a crosslinking agent, e.g., glutaraldehyde, epichlorohydrin, diacrylates, triacrylates, n-acrylates, crosslinkers with 2 or more functional groups for binding to functional groups on starch or amylose. The amount of crosslinking can be used to control a time of biodegradation, with a higher number of crosslinks providing for a longer time required for biodegradation.

An embodiment of the diameter of starch microspheres is from 20 to 300 microns; artisans will immediately appreciate that all ranges and values between the explicitly stated bounds are contemplated, e.g., all the spheres being from 20 to 100 or less than 100 microns diameter and having an average or media diameter from 20-100 microns, or with 20, 30, 40, 50, 60, 70, 80, 90, 100, 110, 120, 150, 190, 200, 250, 290, or 300 being chosen for an endpoint and/or part of a range.

A diameter of a particle or microsphere, unless otherwise indicated, is the MMD. Particles can be characterized in terms of certain properties such as:
D₅₀: Mass-median-diameter (MMD). The log-normal distribution mass median diameter. The MMD is considered to be the average particle diameter by mass.

The embolic materials such as starch beads are non-bioactive, spherical, deformable for delivery through a small catheter and for packing in situ, and may be delivered through a catheter, e.g., a 2.1F-3F catheter. The starch beads are readily suspended in aqueous media so that settling is reduced during delivery. Starch beads are biodegradable to leave only biocompatible residues.

Other embolization particles may be configured and used to provide a transient embolization. An embodiment comprises biodegradable particles consisting of polymeric origin. The term biodegradable refers to a break-down of materials by in vivo causes, be they enzymatic, cellular, or hydrolytic. Hydrolytic degradation (also referred to herein as water-degradable) can be a subcategory of biodegradable and refers to degradation of the links in a polymer or other material by water, e.g., breaking of ester bonds. A particle may be formed so that, upon hydration in physiological solution, a material is formed that is water-degradable, as measurable by the material losing its mechanical strength and eventually dissipating in vitro in an excess of water by hydrolytic degradation of water-degradable groups. This test is predictive of hydrolytically-driven dissolution in vivo, a process that is in contrast to cell or protease-driven degradation. Illustrative water-degradable biodegradable linkages include polymers, copolymers and oligomers of glycolide, dl-lactide, l-lactide, dioxanone, esters, carbonates, and trimethylene carbonate. Illustrative enzymatically biodegradable linkages include peptidic linkages cleavable by metalloproteinases and collagenases. Examples of biodegradable linkages include polymers and copolymers of poly(hydroxy acid)s, poly(orthocarbonate)s, poly(anhydride)s, poly(lactone)s, poly(aminoacid)s, poly(carbonate)s, and poly(phosphonate)s. Further, materials may be used that are biodegradable to leave only biocompatible residues.

The embolic particles or spheres may be made of polymers. Examples of polymers are those of natural and/or certain synthetic materials. Natural materials are those found in nature, including polymers found in nature, and derivatives of the same. Natural polymers include glycosaminoglycans, for example dermatan sulfate, hyaluronic acid, chondroitin sulfates, chitin, heparin, keratan sulfate, keratosulfate, and derivatives thereof. In general, the glycosaminoglycans are extracted from a natural source and purified and derivatized. This modification may be accomplished by various well-known techniques, such as by conjugation or replacement of ionizable or hydrogen bondable functional groups such as carboxyl and/or hydroxyl or amine groups with other more hydrophobic groups. For example, carboxyl groups on hyaluronic acid may be esterified by alcohols to decrease the solubility of the hyaluronic acid. Such processes are used by various manufacturers of hyaluronic acid products to create hyaluronic acid based sheets, fibers, and fabrics that form hydrogels. Other natural polysaccharides, such as carboxymethyl cellulose or oxidized regenerated cellulose, natural gum, agar, agrose, sodium alginate, carrageenan, fucoidan, furcellaran, laminaran, hypnea, eucheuma, gum arabic, gum ghatti, gum karaya, gum tragacanth, locust beam gum, arbinoglactan, pectin, amylopectin, gelatin, hydrophilic colloids such as carboxymethyl cellulose gum or alginate gum.

Natural materials include proteins and peptides. Peptide is a term used herein to refer to a chain of amino acids having no more than 10 residues. Artisans will immediately appreciate that every range and value within these explicit bounds is included, e.g., 1-10, 2-9, 3-10, 1, 2, 3, 4, 5, 6, or 7. Some amino acids have nucleophilic groups (e.g., primary amines or thiols) or groups that can be derivatized as needed to incorporate nucleophilic groups or electrophilic groups (e.g., carboxyls or hydroxyls). Polyamino acid polymers generated synthetically are normally considered to be synthetic if they are not found in nature and are engineered not to be identical to naturally occurring biomolecules.

An advantage of a natural material is that it tends to be available from a cost-effective source and has known biological properties. A disadvantage of such materials is that they can be allergenic or immunogenic. Accordingly, particles may be made that are free of, or essentially free of, amino acids, peptides, proteins, natural materials or any combination of the same. Or the particles may be free of, or essentially free of, allergenic and/or immunogenic materials, (both natural and synthetic materials). Essentially, in this context, means that there is not enough natural material present to be a concern for provoking discomfort in the patient as an allergen/immunogen, e.g., no more than 1 to 10%; Artisans will immediately appreciate that all ranges and values between the explicitly stated bounds are contemplated, with any of the following being available as an upper or lower limit: 1, 2, 3, 4, 5, 6, 7, 8, 9, or 10 percent.

### Embolization Areas

There are a variety of musculoskeletal vasculature that demonstrate hypervascularity in response to chronic inflammation. Transient embolization is an option for them. These musculoskeletal areas include knee (Example 3, for arthritis), rotator cuff (Example 4, for tendinopathy), elbow (Example 5, for lateral epicondylitis), foot (Example 6, for heel pain), shoulder (Example 7, Frozen Shoulder), and knee (Example 8, for patella tendinopathy). Also, certain areas around a stomach may be embolized for ghrelin production control, as in Example 12.

Co-Delivery with Therapeutic Agents

The embolization components may further be co-delivered with therapeutic agents present in the liquid vehicle used to deliver the components and/or in the components themselves, for instance, in embolization beads. Therapeutic agents may be added to treat embolization syndrome, which is a transient effect felt by patients undergoing embolization, with symptoms that include pain and discomfort. This effect is known in uterine artery embolization and other tumor types of embolizations. Agents for co-delivery for treatment of embolization syndrome include analgesics, non-steroidal anti-inflammatory drugs (NSAIDs) and anti-inflammatory agents.

### EXAMPLES

### Example 1: Demonstrating Recanalization of Normal Vasculature 6-24 Hours after Embolization with a Degradable Starch Microsphere

Starch beads EMBOCEPT S (Pharmacept, Berlin) measuring 20-100 m in diameter are diluted using ULTRAVIST 300 Contrast solution (Bayer Healthcare, 300 mgI/mL) to a final bead concentration of 30 mg/mL. This suspension is agitated prior to administration in order to achieve a homogenous suspension. The femoral artery of a New Zealand white rabbit is accessed surgically and a 4F introducer sheath is inserted. A 2.1F single lumen microcatheter is used to track into the kidney via the renal artery. A distal portion of the cranial aspect of the kidney is embolized by the instillation of the starch beads and contrast solution, visualized under X-ray. Complete embolization is achieved and confirmed by performing an angiogram immediately after delivery of the bead suspension. After 6 hours the controlled degradation of the starch beads is complete allowing for recanalization of the vessels. Blood flow has been restored and is confirmed by performing an angiogram, 6 hours after initial delivery of the starch beads.

### Example 2: Demonstrating Permanent Occlusion of Normal Vasculature after Embolization with Permanent or Semi-Permanent Microbeads

EMBOZENE microbeads (Boston Scientific Corporation, Minneapolis), sized 40 μm in diameter, are prepared by diluting 7 mL of material and carrier solution (2 mL microbeads per 7 mL total volume) with ULTRAVIST 300 Contrast solution (300 mgI/mL) for a final bead concentration of 0.18 mL/mL. This suspension is agitated prior to administration in order to achieve a homogenous suspension. The femoral artery of a New Zealand white rabbit is surgically accessed and a 4F introducer sheath is inserted. Then, a 2.1F single lumen microcatheter is used to track into the kidney via the renal artery. A distal portion of the cranial aspect of the kidney is embolized by the instillation of the EMBOZENE microbeads and contrast solution, visualized under X-ray. Complete embolization is confirmed by angiogram. After 3 months, animals may be sacrificed and necrosis of the embolized portion (cranial aspect) of the kidney may be observed. Due to the permanent nature of the nondegradable EMBOZENE microbeads, vessels will not recanalize and necrosis of healthy tissue will occur.

OMNISPHERE microbeads, sized 100 μm in diameter, may be prepared by diluting 7 mL worth of material and carrier solution (2 mL microbeads per 7 mL total volume) with ULTRAVIST 300 Contrast solution (300 mgI/mL) for a final bead concentration of 0.18 mL/mL. This suspension is agitated prior to administration in order to achieve a homogenous suspension. The femoral artery of a New Zealand white rabbit is surgically accessed and a 4F introducer sheath is inserted. Then, a 1.7F single lumen microcatheter is used to track into the kidney via the renal artery. A distal portion of the cranial aspect of the kidney is embolized by the instillation of the OMNISPHERE microbeads and contrast solution, visualized under X-ray. Complete embolization is confirmed by angiogram. After 6 hours, complete and continued occlusion is confirmed by angiogram. Semi-permanent microbeads will degrade completely in 3 months. At 3 months, animals may be sacrificed and necrosis of the embolized portion (cranial aspect) of the kidney may be observed. Due to the semi-permanent nature of the OMNISPHERE microbeads, vessels will not recanalize and necrosis of healthy tissue will occur.

### Example 3: Demonstrating Permanent Occlusion of Abnormal Inflammatory Vasculature Present in Knee Osteoarthritis after Transient Geniculate Artery Embolization (GAE) Using a Degradable Starch Microsphere

Starch beads measuring 20-100 μm in diameter is diluted using Ultravist 300 Contrast solution (300 mgI/mL) to a final bead concentration of 30 mg/mL. This suspension may be agitated prior to administration in order to achieve a homogenous suspension. A bed of abnormal inflammatory vasculature resulting in the diagnosis of knee osteoarthritis in a patient is visualized by angiogram, appearing as a vascular blush off of the main geniculate artery. The area is accessed selectively using a 2.1F single lumen microcatheter over a 0.014″ guide wire. The starch beads are delivered on target and embolization of the abnormal vessels is confirmed by angiogram using contrast. After the known degradation period of 3 hours, the inflammatory vasculature remains occluded and complete embolization of this area will have been maintained, confirmed by angiogram. This therapy decreases or eliminates abnormal neovessels, decreases local tenderness, and decreases in arterial flow on target lesion. This approach is unique due to the predictability of the degradation period of the starch beads, and unlike the unpredictability of other embolics like lipiodol or non-degradable microspheres.

### Example 4: Demonstrating Permanent Occlusion of Abnormal Disorganized Hypervasculature Present in Rotator Cuff Tendinopathy after Transient Transcatheter Arterial Embolization (TAE) Using a Degradable Starch Microsphere

Starch beads measuring 20-100 μm in diameter are diluted using ULTRAVIST 300 Contrast solution (300 mgI/mL) to a final bead concentration of 30 mg/mL. This suspension is agitated prior to administration in order to achieve a homogenous suspension. Abnormal hypervasculature around the shoulder is identified by angiogram. The area is accessed selectively using a 2.1F single lumen microcatheter over a 0.014″ guide wire. The starch beads are delivered on target and embolization is confirmed by angiogram using contrast. After the known degradation period of 3 hours, flow will not have been restored and complete embolization of this area will have been maintained, as may be confirmed by angiogram. This therapy will result in a decrease in arterial flow to the hypervascular site and prevent further tissue degeneration. Additionally, a change from baseline will be observed in various clinical parameters including visual analog scale pain scale and decrease or elimination of conventional treatment such as pain relievers or corticosteroid injections. This approach is unique due to the predictability of the degradation period of the starch beads, and unlike the unpredictability of other embolics like lipiodol or non-degradable microspheres. Adverse events likely with permanent embolics such as skin necrosis/color change, peripheral paresthesia/numbness, and muscle weakness/dull pain my not occur with transient embolization.

### Example 5: Demonstrating Permanent Occlusion of Abnormal Inflammatory Vasculature Present in Lateral Epicondylitis after Transient Transcatheter Arterial Embolization (TAE) Using a Degradable Starch Microsphere

Starch beads measuring 20-100 μm in diameter re diluted using ULTRAVIST 300 Contrast solution (300 mgI/mL) to a final bead concentration of 30 mg/mL. This suspension is agitated prior to administration in order to achieve a homogenous suspension. A bed of abnormal inflammatory vasculature present in lateral epicondylitis is visualized by angiogram, appearing as a vascular blush off of the main artery. The area is accessed selectively using a 2.1F single lumen microcatheter over a 0.014″ guide wire. The starch beads are delivered on target and embolization is confirmed by angiogram using contrast. After the known degradation period of 3 hours, flow will not have been restored and complete embolization of this area will have been maintained, as may be confirmed by angiogram. This therapy will result in a decrease or elimination of abnormal neovessels, decrease in local tenderness, and decrease in arterial flow on target lesion. Additionally, a change from baseline will be observed in various clinical parameters including visual analog scale pain score, Patient-Rated Tennis Elbow Evaluation score, and pain free grip strength. This approach is unique due to the predictability of the degradation period of the starch beads, and unlike the unpredictability of other embolics like lipiodol or non-degradable microspheres. Adverse events likely with permanent embolics such as skin necrosis/color change, peripheral paresthesia/numbness, and muscle weakness/dull pain my not occur with transient embolization.

### Example 6: Demonstrating Permanent Occlusion of Abnormal Inflammatory Vasculature Present in Heel Pain after Transient Transcatheter Arterial Embolization (TAE) Using a Degradable Starch Microsphere

Starch beads measuring 20-100 μm in diameter may be diluted using ULTRAVIST 300 Contrast solution (300 mgI/mL) to a final bead concentration of 30 mg/mL. This suspension may be agitated prior to administration in order to achieve a homogenous suspension. A bed of abnormal inflammatory vasculature present in heel pain is visualized by angiogram, appearing as a vascular blush off of the main posterior tibial artery. The area will be accessed selectively using a 2.1F single lumen microcatheter over a 0.014″ guide wire. The starch beads will be delivered on target and embolization will be confirmed by angiogram using contrast. After the known degradation period of 3 hours, flow will not have been restored and complete embolization of this area will have been maintained, confirmed by angiogram. This therapy will result in a decrease or elimination of abnormal neovessels, decrease in local tenderness, and decrease in arterial flow on target lesion. Additionally, this therapy may also improve gait. This approach is unique due to the predictability of the degradation period of the starch beads, and unlike the unpredictability of other embolics like lipiodol or non-degradable microspheres. Adverse events likely with permanent embolics such as skin necrosis/color change, peripheral paresthesia/numbness, and muscle weakness/dull pain my not occur with transient embolization.

### Example 7: Demonstrating Permanent Occlusion of Abnormal Inflammatory Vasculature Present in Adhesive Capsulitis after Transient Transcatheter Arterial Embolization (TAE) Using a Degradable Starch Microsphere

Starch beads measuring 20-100 μm in diameter are diluted using ULTRAVIST 300 Contrast solution (300 mgI/mL) to a final bead concentration of 30 mg/mL. This suspension is agitated prior to administration in order to achieve a homogenous suspension. A bed of abnormal inflammatory vasculature present in adhesive capsulitis is visualized by angiogram, appearing as a vascular blush off of the main artery at the rotator interval. The area is accessed selectively using a 2.1F single lumen microcatheter over a 0.014″ guide wire. The starch beads are delivered on target and embolization is confirmed by angiogram using contrast. After the known degradation period of 3 hours, flow will not have been restored and complete embolization of this area will have been maintained, as may be confirmed by angiogram. This therapy results in a decrease or elimination of abnormal neovessels, a decrease in local tenderness, and a decrease in arterial flow on target lesion. This approach is unique due to the predictability of the degradation period of the starch beads, and unlike the unpredictability of other embolics like lipiodol or non-degradable microspheres. Adverse events likely with permanent embolics such as skin necrosis/color change, peripheral paresthesia/numbness, and muscle weakness/dull pain may not occur with transient embolization.

### Example 8: Demonstrating Permanent Occlusion of Abnormal Inflammatory Vasculature Present in Patella Tendonopathy after Transient Transcatheter Arterial Embolization (TAE) Using a Degradable Starch Microsphere

Starch beads measuring 20-100 μm in diameter are diluted using ULTRAVIST 300 Contrast solution (300 mgI/mL) to a final bead concentration of 30 mg/mL. This suspension is agitated prior to administration in order to achieve a homogenous suspension. A bed of abnormal inflammatory vasculature associated with tendonopathy is visualized by angiogram, appearing as a vascular blush of the main geniculate artery. The area is accessed selectively using a 2.1F single lumen microcatheter over a 0.014″ guide wire. The starch beads is delivered on target and embolization is confirmed by angiogram using contrast. After the known degradation period of 3 hours, flow will not have been restored and complete embolization of this area will have been maintained, as may be confirmed by angiogram. This therapy results in a decrease or elimination of abnormal neovessels, a decrease in local tenderness, and a decrease in arterial flow on target lesion. This approach is unique due to the predictability of the degradation period of the starch beads, and unlike the unpredictability of other embolics like lipiodol or non-degradable microspheres. Adverse events likely with permanent embolics such as skin necrosis/color change, peripheral paresthesia/numbness, and muscle weakness/dull pain my not occur with transient embolization.

### Example 9: Enzymatic Degradation of Starch Beads Using Biological Solution of Saliva

EMBOCEPT S starch beads degrade via enzymatic degradation by amylase, an enzyme found chiefly in saliva and pancreatic fluid. This enzyme converts the starch and glycogen into simple sugars. 10 mL of a stock solution of EMBOCEPT S starch beads at 60 mg/mL are added to a 20 mL scintillation vial. At t=0 hr, a sample of saliva biological solution is added to the vial, at which point a stop watch is started. Over time, the enzymatic degradation of the starch beads is visualized as the suspension became a homogenous solution with no obvious particulates. Complete degradation is confirmed by pre/post weights of a dry filter paper after filtering the solution.

### Example 10: Co-Delivering Lidocaine with Degradable Starch Beads

2 mL of 60 mg/mL starch beads measuring 20-100 μm in diameter are diluted to a concentration of 30 mg/mL using 2 mL of a 2% solution of Lidocaine. Then, the suspension is further diluted using ULTRAVIST 300 Contrast solution (300 mgI/mL) to a final concentration of beads of 15 mg/mL. This suspension is agitated prior to administration in order to achieve a homogenous suspension. A bed of abnormal inflammatory vasculature of a patient is visualized by angiogram, appearing as a vascular blush off of a main arterial vessel. The area is accessed selectively using a 2.1F single lumen microcatheter over a 0.014″ guide wire. The starch beads is delivered on target and embolization is confirmed by angiogram using contrast. The lidocaine provides an anesthetic effect in order to quell some of the transient pain associated with vessel closure during embolization (post embolization syndrome). After the known degradation period of 3 hours, there will be no recanalization of the inflammatory vasculature and flow will be blocked to the fine inflammatory vasculature, while the feeder artery continues to be patent. This can be confirmed by angiogram.

### Example 11: Co-Delivering Dexamethasone Sodium Phosphate with Degradable Starch Beads

2 mL of 60 mg/mL starch beads measuring 20-100 μm in diameter are diluted to a concentration of 30 mg/mL using 2 mL of a 1% solution of Dexamethasone Sodium Phosphate. Then, the suspension can be further diluted using ULTRAVIST 300 Contrast solution (300 mgI/mL) to a final concentration of beads of 15 mg/mL. This suspension may be agitated prior to administration in order to achieve a homogenous suspension. A bed of abnormal inflammatory vasculature of a patient is visualized by angiogram, appearing as a vascular blush off of a main arterial vessel. The area will be accessed selectively using a 2.1F single lumen microcatheter over a 0.014″ guide wire. The starch beads will be delivered on target and embolization will be confirmed by angiogram using contrast. The dexamethasone will provide an anti-inflammatory effect in order to help treat and prevent inflammation associated with the disease state as well as vessel closure during embolization (post embolization syndrome). After the known degradation period of 3 hours, there will be no recanalization of the inflammatory vasculature and flow will be blocked to the fine inflammatory vasculature, while the feeder artery continues to be patent, which can be confirmed by angiogram.

### Example 12: Conjugated Delivery Techniques of Starch Beads and EMBOZENE Beads in a Bariatric Setting

A conjugated delivery of permanent and transient embolics is performed as follows. EMBOZENE embolic beads (>100 μm) are introduced using a 2.8F catheter system delivered to target larger normal vasculature feeding the fundus portion of the stomach, responsible for the production of ghrelin, for permanent embolization. After embolization the larger, less selective catheter system is removed. For the follow up transient embolization, starch beads measuring 20-100 μm in diameter are diluted using ULTRAVIST 300 Contrast solution (300 mgI/mL) to a final concentration of beads of 30 mg/mL. This suspension is agitated prior to administration in order to achieve a homogenous suspension. The interventional radiologist (IR) would access the desired location in the gastric artery feeding the fundus portion of the stomach, using a selective 2.1F microcatheter. Starch beads will be delivered to embolize the finer vasculature and effectively halt flow into the finer vaculature. The starch beads degrade in a predictable and controlled fashion by design after which flow will have been shut down due to the inability of abnormal vasculature to recanalize. This can be confirmed by angiogram. This would be an effective way to provide combined transient and permanent embolization or conjugated treatment to the fundus region of the stomach as an interventional approach to treat obesity.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to measurement error and/or reporting precision as would be understood by a person of ordinary skill in the art in the context for the particular parameter unless explicitly indicated otherwise.

What is claimed is:

1. A method of embolizing a hypervascular vessel formed in response to chronic inflammation in a musculoskeletal vasculature, the method comprising:
advancing a catheter through a vasculature to a parent artery; and
releasing an embolic material from a distal end of the catheter into the hypervascular vessel, with the embolic material blocking blood flow in the hypervascular vessel without blocking blood flow to a parent vessel, wherein the embolic material is free of a therapeutic agent and comprises deformable hydrogel particles that are biodegradable within a period of time from 15 minutes to 48 hours.

2. The method of claim 1 wherein the particles have an average diameter from about 20 microns to about 300 microns.

3. The method of claim 1 wherein the particles have an average diameter from about 20 microns to about 250 microns.

4. The method of claim 1 wherein the particles are substantially spherical.

5. The method of claim 1 wherein the particles are of polymeric origin.

6. The method of claim 1 wherein the particles comprise gelatin.

7. The method of claim 1 wherein the particles comprise crosslinked gelatin.

8. The method of claim 1 wherein the particles are hydrolytically degradable.

9. The method of claim 1 wherein the particles are biodegradable by enzymatic action.

10. The method of claim 1 wherein the particles are biodegradable by collagenase.

11. The method of claim 1 wherein the particles are biodegradable to biocompatible residues.

12. The method of claim 1 wherein the embolic material further comprises a contrast agent.

13. The method of claim 1 wherein the period of time is from 15 minutes to 8 hours.

14. The method of claim 1 being a treatment for an area related to a heel, spine, shoulder, hip, knee, or elbow.

15. A medical system for treatment of a hypervascular vessel formed in response to chronic inflammation in a musculoskeletal vasculature, the medical system comprising:
a catheter suitable for delivery through a patient's vasculature to reach the hypervascular vessel; and
a delivery component comprising a reservoir of the embolic material configured for delivery through the catheter, wherein the embolic material is free of a therapeutic agent and comprises deformable hydrogel particles that are biodegradable within a period of time from 15 minutes to 48 hours.

16. The medical system of claim 15 wherein the delivery component comprises a syringe or pump.

17. The medical system of claim 15 wherein the catheter comprises a 1.7F to 3F catheter.

18. The medical system of claim 15 wherein the particles comprise gelatin.

19. The medical system of claim 15 wherein the particles are hydrolytically degradable.

20. The medical system of claim 15 wherein the particles are biodegradable by enzymatic action.
